(19) United States
(12) Patent Application Publication
(10) Pub. No.: US 2015/0150509 A1
(43) Pub. Date: Jun. 4, 2015

(54) MEDICAL DIAGNOSTIC APPARATUS

(71) Applicant: CANON KABUSHIKI KAISHA, Tokyo (JP)

(72) Inventor: Yohei Motoki, Yokohama-shi (JP)

(21) Appl. No.: 14/405,255

(22) PCT Filed: Jun. 25, 2013
(86) PCT No.: PCT/JP2013/003948

§ 371 Date: Dec. 3, 2014

(30) Foreign Application Priority Data
Jul. 13, 2012 (JP) 2012-157697

(51) Int. Cl. A61B 5/00; A61B 8/08; A61B 6/00

(52) U.S. Cl. CPC A61B 5/708; A61B 5/0095; A61B 6/502; A61B 8/0825; A61B 5/4312

(57) ABSTRACT

There is used a medical diagnostic apparatus having a bed that supports an examinee in a prone position, and has a first opening in which an object being a part of the examinee is inserted, a measurement unit that has a second opening which is smaller than the first opening and in which the object is inserted through the first opening, a moving mechanism that moves the bed and the measurement unit relative to each other, and a shock absorbing member that is disposed between the bed and the examinee. The shock absorbing member has a portion covering a part of the first opening at least in a direction where the bed and the measurement unit move relative to each other, and is arranged such that the covering portion does not cover the second opening.

### TECHNICAL FIELD

The present invention relates to a medical diagnostic apparatus.

### BACKGROUND ART

There is a breast inspection apparatus including a bed on which an examinee is placed in a prone position, wherein a breast of the examinee is inserted into a hole provided in the bed. Examples of such a breast inspection apparatus include, for example, an apparatus in which the inserted breast is held and pressed between a breast press plate made of a material which an X-ray penetrates, and an imaging plate including a sensor. An examinee which lies prone on a bed of such an apparatus inserts her breast from a breast insertion opening provided in the bed so that the breast hangs down. Then, an operator or the like tucks the hanging breast by the breast press plate, and applies an X-ray to take an image. This is because the examinee is not forced into a stress position and imaging is performed in a relaxed state, so that accurate measurement is made while suppressing body motion.

It is considered that unless positioning of the breast press plate and the breast is precisely performed, a region desired to be imaged deviates from an irradiation region of an X-ray, or a distance between the fixed press plate and the breast increases. As a result, there is a risk that an effective image for an examination is unable to be obtained, or a necessary region is unable to be imaged. In this case, since positioning is performed again after the examinee moves and inserts her breast into the insertion opening again, the burden of the examinee is increased.

PTL 1 proposes a method of moving a bed supporting an examinee relative to a fixed press plate, or a movable holding plate. Consequently, accuracy of the positioning of the breast can be improved without placing a burden of movement on the examinee.

### CITATION LIST

### Patent Literature

### [PTL 1]

### Japanese Patent Application Laid-open No. 2011-240116

### SUMMARY OF INVENTION

### Technical Problem

When positioning of a breast is performed by a medical diagnostic apparatus in which an examinee is in a prone position, hanging tissues such as ribs of the examinee may be superimposed on an object at the time of imaging. Therefore, in order to obtain an effective diagnostic image, a member for preventing the tissues from hanging preferably supports a chest wall. However, in a case where the bed and a housing are moved relative to each other in positioning as in PTL 1, there is a possibility that an edge of an opening or a chest wall supporting member is pressed against, or collides with the examinee in a prone position. Furthermore, there is a possibility that such a member causes difference in level, and gives the examinee an oppressive feeling. Thus, a conventional prone position type medical diagnostic apparatus has room for improvement in terms of suppressing the burden of the examinee and enhancing comfort.

The present invention has been conceived in order to solve the aforementioned problems, and the object thereof is to provide a technique for suppressing the burden of an examinee in a medical diagnostic apparatus in which the examinee is in a prone position on a bed.

### Solution to Problem

The present invention provides a medical diagnostic apparatus comprising:

a bed configured to support an examinee in a prone position, and have a first opening in which an object being a part of the examinee is inserted;

a measurement unit configured to have a second opening, which is smaller than the first opening and in which the object is inserted through the first opening;

a moving mechanism configured to move the bed and the measurement unit relative to each other; and

a shock absorbing member configured to be disposed between the bed and the examinee, wherein

the shock absorbing member has a portion covering a part of the first opening at least in a direction where the bed and the measurement unit move relative to each other, and is arranged such that the covering portion does not cover the second opening.

### Advantageous Effects of Invention

According to the present invention, it is possible to provide a technique for suppressing the burden of an examinee in a medical diagnostic apparatus in which the examinee is in a prone position on a bed.

Further features of the present invention will become apparent from the following description of exemplary embodiments with reference to the attached drawings.

### BRIEF DESCRIPTION OF DRAWINGS

FIG. 1 is a schematic diagram showing a configuration of a medical diagnostic apparatus of the present invention.

FIG. 2A and FIG. 2B are schematic diagrams showing an example of a shock absorbing member of the present invention.

FIG. 3A and FIG. 3B are schematic diagrams showing an example of a measurement unit of the present invention.

FIG. 4A to FIG. 4C are schematic diagrams showing an example of arrangement of a shock absorbing member of the present invention.

### DESCRIPTION OF EMBODIMENTS

Hereinafter, a preferred embodiment of the present invention will be described with reference to the drawings. However, size, quality of materials, shape, relative arrangement of components described below should be appropriately changed according to a configuration of an apparatus to which the invention is applied, or various conditions, and the scope of this invention is not limited to the description as below.

The present invention is applicable to medical diagnostic apparatuses utilized by an examinee lying in a prone position. Examples of the medical diagnostic apparatuses include an apparatus utilizing an X-ray, an ultrasound diagnostic apparatus utilizing an ultrasound echo to acquire difference in acoustic impedance, a photoacoustic diagnostic apparatus utilizing a photoacoustic effect, and the like. In the present embodiment, the photoacoustic diagnostic apparatus is described as an example.

The photoacoustic diagnostic apparatus is an apparatus receiving an acoustic wave generated inside an object by a photoacoustic effect when applying light (electromagnetic wave) to the object, and acquiring characteristic information inside the object as image data. The characteristic information acquired at this time indicates a generation source distribution of acoustic waves generated by light irradiation, or an initial sound pressure distribution inside the object, an optical energy absorption density distribution or an absorption coefficient distribution derived from the initial sound pressure distribution, or a concentration distribution of substances configuring a tissue. The concentration distribution of substances includes an oxygen saturation distribution, an oxygenated/reduced hemoglobin concentration distribution, or the like, for example.

An acoustic wave in the present invention is typically an ultrasound wave, and includes an elastic wave referred to as a sound wave, an ultrasound wave or an acoustic wave. An acoustic wave generated by a photoacoustic effect is referred to as a photoacoustic wave, or a light-induced ultrasound wave. This photoacoustic wave is received by an acoustic wave probe, and characteristic information is acquired.

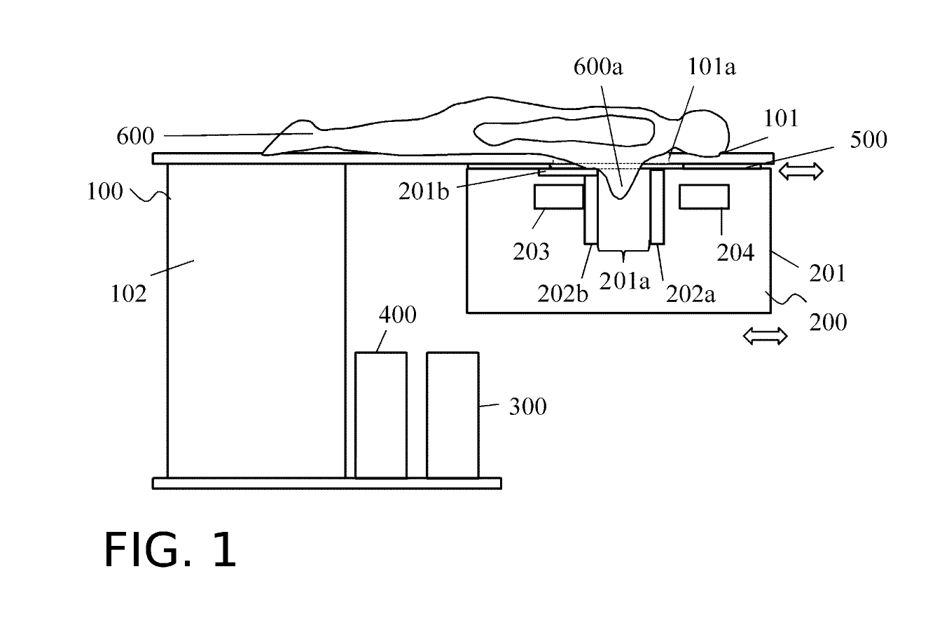

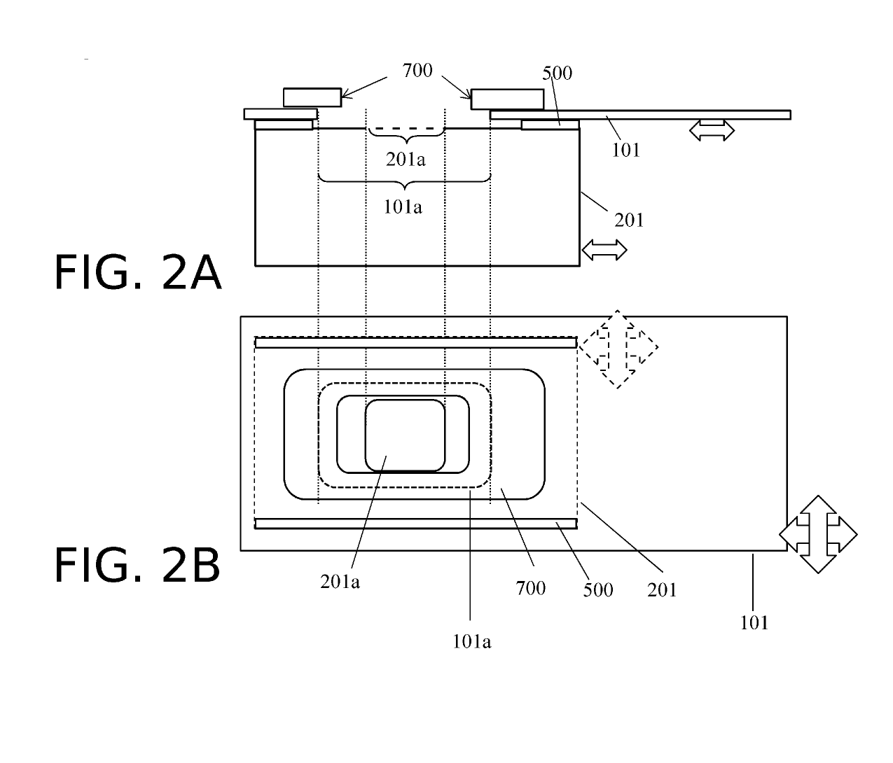

Hereinafter, an embodiment of the present invention will be described with reference to FIG. 1 and FIG. 2. FIG. 1 is a schematic diagram showing an example of a configuration of a photoacoustic diagnostic apparatus. FIG. 2 is a schematic diagram showing an example of a shock absorbing member (compliance member), in which FIG. 2A shows a side elevational view, and FIG. 2B shows a top plan view.

An examinee 600 lies in a prone position on a bed 101, and inserts an object 600a being a part of the examinee from a first opening 101a being an opening of the bed 101. The object 600a is further inserted in a second opening 201a being an opening of a housing 201 through the first opening 101a. The object of the present embodiment is a breast, but is not limited to this.

Here, there is a possibility that an image cannot suitably taken depending on a position where the object 600a is inserted. For example, the image cannot be suitably taken in a case where a position of an imaging region of the breast is deviated from a light irradiation position by an irradiation unit, or from an acoustic wave receiving position by a probe. Therefore, positioning is performed by a moving mechanism unit 500 such that the object 600a is located at a suitable position. The moving mechanism unit 500 may be provided at any location as long as the bed 101 and the housing 201 can move relative to each other. For example, the moving mechanism unit 500 can be provided on a side of the bed 101, a side of the housing 201, or both sides thereof.

The object 600a is inserted between a movable holding plate 202a and a fixed holding plate 202b, and sandwiched by movement of the movable holding plate 202a. When an end of the object 600a is struck against the fixed holding plate 202b by the relative movement of the bed 101 and the housing 201 before the movement of movable holding plate 202a, the object 600a can be stably sandwiched.

After the object is held, light (pulsed light) generated by the light source unit 300 is applied to the object 600a through the movable holding plate 202a by an irradiation unit 204. Alight absorber (object to be detected such as a tumor) inside the object, irradiated with the light absorbs light energy, and generates an acoustic wave by a photoacoustic effect. The generated acoustic wave propagates inside the object to reach an acoustic wave probe 203 through the fixed holding plate 202b.

The acoustic wave probe 203 receives the acoustic wave to converts the same to an electric signal, and outputs the electric signal to the arithmetic processor 400. The arithmetic processor 400 performs signal processing, or controls respective segments inside the apparatus. The arithmetic processor 400 generates characteristic information inside the object by using the input electric signal, generates photoacoustic image data on the basis of the characteristic information, and displays the image on a display unit (not shown).

However, in a case where the bed and the housing are moved relative to each other in the positioning, there is a possibility of giving the examinee an oppressive feeling or stimulation caused by collision. Additionally, there is a possibility that an edge of the opening or a chest wall supporting member causes difference in level and is pressed against the examinee in the prone position. Therefore, there is room for improvement in terms of suppressing the burden of the examinee and enhancing comfort.

A clearance exist between the bed 101 and the housing 201, and therefore it cannot be said that there is no risk of catching a part of a body of the examinee 600 in the clearance between the bed 101 and the housing 201 at all when the positioning is performed by the moving mechanism unit 500.

In the present invention, a shock absorbing member (compliance member) 700 is provided in order to suppress the burden of the examinee and avoid the catching. The shock absorbing member (compliance member) 700 is arranged between the bed 101 and the examinee 600 at least in a direction where the bed 101 and the housing 201 move relative to each other. A range of the arrangement is set so as to cover at least an edge of the first opening 101a. The shock absorbing member (compliance member) 700 is placed so as to have a portion (portion covering a part of the first opening 101a) spread into the first opening 101a of the bed when projected on the bed 101. This spread portion (covering portion) covers the difference in level, or the clearance between the bed 101 and the housing 201 when the object 600a is inserted. Additionally, a spread amount is set to such a length that the shock absorbing member (compliance member) 700 does not cover the second opening 201a when projected on the housing 201.

When the examinee 600 takes the prone position in this state, the shock absorbing member (compliance member) 700 hangs by a weight of the examinee 600 from the first opening 101a of the bed 101, and fills the clearance between the bed 101 and the housing 201. In other words, the shock absorbing member 700 (compliance member) contacts the housing 201. Consequently, it is possible to suppress an influence of oppression due to the difference in level or collision due to the relative movement on the examinee 600, to reduce the burden, and to enhance comfort. Additionally, a part of the body of the examinee 600 can be prevented from being tucked or caught at the time of the relative movement.

The spread amount of the shock absorbing member (compliance member) 700 is set to such an amount of 10 mm or more as not to enter inside the second opening 201a, as an example. Consequently, the shock absorbing member (compliance member) 700 can secure a hanging amount for filling the clearance between the bed 101 and the housing 201, and can be further prevented from hanging inside the second opening 201a to interfere with the imaging region. The spread amount is adjusted by a distance between the housing and the bed, or a configuration of the moving mechanism unit.

Hereinafter, a specific configuration of the photoacoustic diagnostic apparatus will be described.

Abet unit 100 supports the examinee 600 in the prone position, and is configured by the bed 101, and a bed supporting post 102 which supports the bed 101. The bed 101 is provided with the first opening 101a in which the object 600a being a part of the examinee 600 is inserted. The first opening 101a enables insertion of the object 600a, and is larger than the second opening 201a of the housing 201. As long as such requirements are met, the first opening 101a may have any shape.

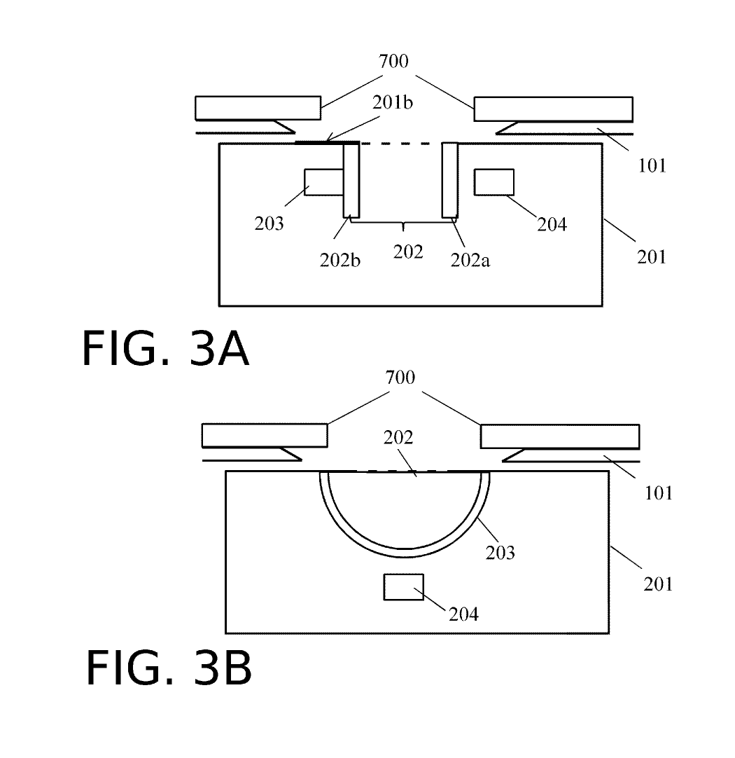

FIG. 3 is a schematic diagram showing an example of the measurement unit. A measurement unit is configured by the housing 201, a holding unit 202, the acoustic wave probe 203, and the irradiation unit 204. FIG. 3A shows a case where the holding unit 202 is a parallel plate type, and FIG. 3B shows a case where the holding unit 202 is a film member placed on the second opening.

The housing 201 has the second opening 201a for inserting the object of the examinee. The housing 201 preferably has a configuration in which light is blocked from places other than the first opening 101a. A metal material such as aluminum, magnesium and iron, which does not transmit a laser, is preferably used for the housing 201. Alternatively, the laser can be blocked also by attaching a metal film to carbon, resin or the like. As long as the second opening 201a has such size that the object 600a can be inserted, the second opening 201a may have any shape.

Additionally, the second opening 201a is preferably provided with a chest wall supporting member 201b on a foot side of the examinee 600 taking the prone position. The chest wall supporting member 201b is provided in order to support a load of a chest wall portion of the examinee 600. The chest wall supporting member 201b is preferably a material such as tungsten carbide, which is hard, has high Young's modulus, and is unlikely to bend.

Here, the box-shaped housing 201 configures outer walls of the measurement unit. While the box-shape is preferable from the standpoint of blocking light, the mechanism which stores respective components of the measurement unit is not limited to this. As long as the mechanism is in the form of plates, and has an opening for inserting the object 600a through the first opening 101a, the present invention is applicable.

In FIG. 3A, a holding unit 202 includes a movable holding plate 202a and a fixed holding plate 202b that are two plate-like members, and the object is sandwiched between these two holding members. In FIG. 3B, a holding unit 202 has a thin film-like holding member on a second opening 201a, and holds a breast in a hanging direction. As long as the holding unit has such a configuration as to enable to hold the object, apply light, and receive the acoustic wave, any holding unit may be employed.

In the case of FIG. 3A, at least a partial shape of the object 600a can be kept constant. Additionally, the object 600a is sandwiched from both sides thereof to be fixed, and hence an influence of body motion of the examinee 600 or the like can be reduced. Furthermore, a thickness of the object 600a can be reduced by shortening a distance between the holding plates, and hence light can be effectively reach a depth portion.

As the movable holding plate 202a provided on a side where an irradiation unit 204 is provided, a member having high light transmittance is preferable. As the fixed holding plate 202b provided on a side where an acoustic wave probe 203 is provided, a member having acoustic consistency with the object or the acoustic wave probe 203 is preferable. For the purpose of enhancing the acoustic consistency, an acoustically suitable material such as gel may be interposed between the fixed holding plate 202b and the object 600a.

The positional relation between the fixed holding plate 202b and the movable holding plate 202a is not limited to that shown in FIG. 3A, and the irradiation unit 204 side may be fixed, or both sides may be movable. Furthermore, a position where light is applied or an acoustic wave is received can be set to various positions. The irradiation unit 204 and the acoustic wave probe 203 are configured to enable scanning on the holding plate, thereby enabling a wide range of measurement.

The acoustic wave probe is provided on the fixed holding plate side in FIG. 3A, but may be provided on the movable holding plate side.

In the case of FIG. 3B, a holding film member is placed on the second opening 201a, and supports the hanging object 600a. The shape of the holding film member may be planar, or molded to have a breast shape. As the holding film member, a member excellent in light transmittance and having high acoustic consistency with the object is preferable. Additionally, the holding film member is preferably placed on the second opening 201a, but is not limited to this. An acoustically suitable material such as gel may be interposed between the holding film material and the acoustic wave probe 203.

The acoustic wave probe 203 has at least one element that receives an acoustic wave to convert the same to an electric signal. As long as the element can receive an acoustic wave to convert the same to an electric signal, any element such as a transducer using a piezoelectric effect, a transducer using resonance of light, a transducer using change in capacity may be employed. A plurality of the elements receiving an acoustic wave are arranged one-dimensionally or two-dimensionally, thereby enabling the acoustic wave to be simultaneously received at a plurality of places, enabling receiving time to be shortened and enabling an influence such as vibration of the object to be reduced. It is also possible to obtain a signal similar to that obtained when the plurality of elements are arranged one-dimensionally or two-dimensionally, by moving a single element.

In FIG. 3B, the elements may not be arranged on the same plane, but may be arranged on a curved surface such a bowl shape. In this case, angle dependence can be reduced when characteristic information is reconfigured on the basis of an acoustic wave generated from the object 600a.

An irradiation unit 204 has an optical system such as a mirror reflecting light, a half mirror for branching into reference light and irradiation light, a lens collecting and expanding light to change a shape, and an optical waveguide, for example. As long as the optical system can apply light, which is generated from a light source, in a desired shape, any optical system may be employed. An area of light is preferably expanded to a certain extent by diffusing light with the lens. Consequently, safety standards are satisfied, and efficiency of measurement can be improved. Additionally, a region where light is applied to the object is preferably movable on the object, in order to apply light in a wide range. Examples of a method of moving the region where the light is applied to the object include a method of using a movable mirror or the like, a method of mechanically moving the light source, and the like. The irradiation unit 204 may be integral with the holding unit 202.

Alight source unit 300 is configured by a light source unit including at least one coherent or incoherent pulsed light source, and an optical transmission unit transmitting to the irradiation unit a light source which is output from the light source unit. In order to generate an photoacoustic effect, the light source unit preferably has a pulse width of several hundred nanoseconds or less, and more preferably has a pulse width of 5 nsec to 50 nsec. Additionally, in a case of measuring a breast cancer or the like, light with a specific wavelength, which is absorbed in a specific constituent (e.g., hemoglobin) among constituents configuring a biological body, is applied. Specifically, a wavelength of at least 500 nm and not more than 1200 nm is preferable. As the light source, a laser which obtains large output is preferable, but a light-emitting diode or the like can be employed in place of the laser. As the laser, various lasers such as a solid-state laser, a gas laser, a dye laser, and a semiconductor laser can be used. For example, Nd:YAG laser or Ti:sapphire laser can be used. Additionally, the wavelength may be variable. As long as the optical transmission unit can effectively transmit light, any optical transmission unit may be employed. For example, light may be transmitted in a space by using a mirror, or transmitted in a fiber by using a light fiber.

The arithmetic processor 400 performs processing of calculating a light irradiation density distribution, and giving feedback to irradiation light from the result, by software previously programmed. Additionally, the arithmetic processor 400 performs noise reduction processing or the like for an electric signal taken from the acoustic wave probe. Furthermore, the arithmetic processor 400 performs general processing of operating the photoacoustic diagnostic apparatus, such as control of a scanning mechanism. As the arithmetic processing unit 400, a workstation is typically used.

As long as the moving mechanism unit 500 has a mechanism capable of moving the bed and the measurement unit relative to each other, any moving mechanism unit may be employed, and should be provided on at least one of the bed and the measurement unit. In a case of linear motion, the moving mechanism unit 500 is configured by a linear motion mechanism such as a ball screw, a linear guide, a chain, a timing belt, and the like. In a case of turning motion, the moving mechanism unit 500 is configured by a turning motion mechanism like a thrust bearing, an R guide, or a cross roller bearing. The moving mechanism unit can combine linear motion and turning motion according to motion desired as the apparatus. A power transmission unit may be electrically or manually powered. In a case of electric operation, a DC motor or a blushless motor is used. Ina case of manual operation, the power transmission unit is used along with such a mechanism as to convert hand motion into power, such as a handle or a grip.

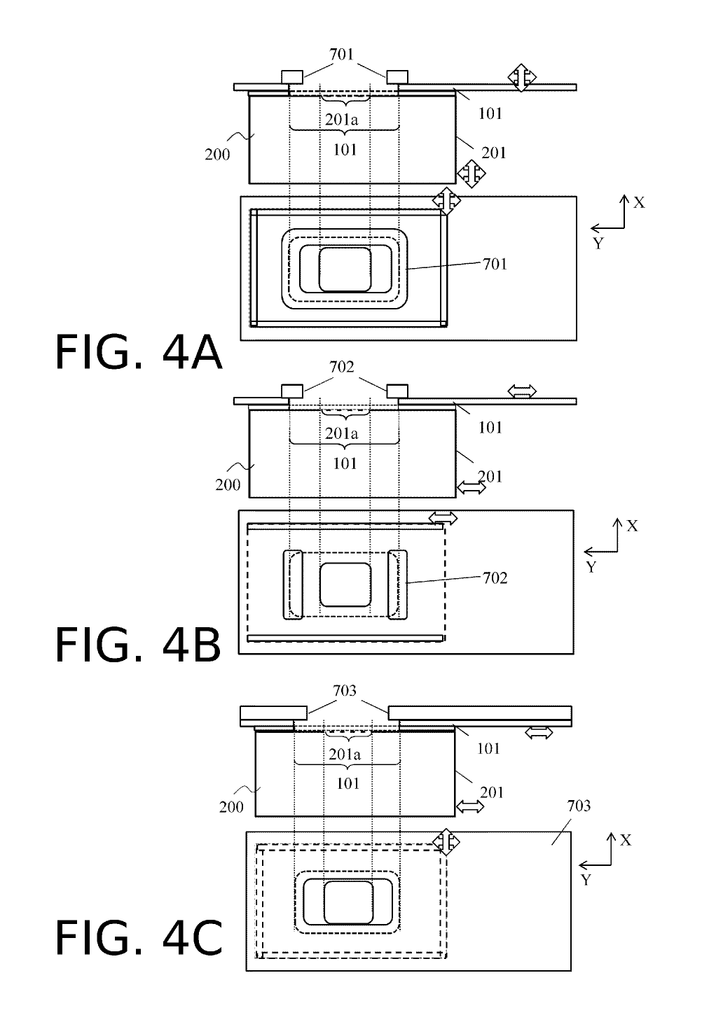

FIG. 4 shows arrangement examples of the shock absorbing member (compliance member).

The shock absorbing member (compliance member) is arranged at least in the direction where the bed and the housing move relative to each other. A range of the arrangement is set so as to cover at least the edge of the first opening 101a. The shock absorbing member (compliance member) has a portion spread into the first opening when projected on the bed. This spread amount is set to such a length that the shock absorbing member (compliance member) does not cover the second opening. The spread amount is preferably set to 10 mm or more.

FIG. 4A is an example in which the bed 101 and the housing 201 move relative to each other in an XY direction. In this case, a shock absorbing member (compliance member) 701 is arranged so as to cover an edge of the first opening in the XY direction. Thus, the shock absorbing member (compliance member) 701 spreads on a whole region of the first opening, thereby enabling the shock absorbing member (compliance member) 701 to correspond to all of the relative movement of the bed and the measurement unit. The first and second openings are formed in various shapes such as a rectangle and a circle.

FIG. 4B is an example in which the bed 101 and the housing 201 move relative to each other only in a Y direction. In this case, a shock absorbing members (compliance member) 702 cover both ends of an edge in the Y direction of the first opening. The spread portions of the shock absorbing members (compliance member) cover the moving mechanism unit or the first opening in a movement direction, thereby enabling reduction of the burden of the examinee, and prevention of catching.

FIG. 4C is an example of the shape of the shock absorbing member (compliance member) or an arranged segment. Here, a shock absorbing member (compliance member) 703 is arranged over the whole of the bed 101. Consequently, the whole body of the examinee can be supported, and a burden can be further reduced.

When the shock absorbing member (compliance member) is arranged, even the examinee moves in a state of the prone position, the shock absorbing member (compliance member) is preferably fixed such that a position thereof is not deviated. For example, a method of fixing the shock absorbing member (compliance member) to the bed by attaching double-sided tape or a double-sided fastener can be utilized. Additionally, in order to improve reproducibility of the position where the shock absorbing member (compliance member) is fixed, the position is previously marked on the bed.

The shock absorbing member (compliance member) is preferably made of such a material that permanent strain does not remain even after applying a load for a long time, and that the member is not completely crushed. For example, expanded polystyrene foam, polyester elastomer, or the like can be utilized. A thickness of the shock absorbing member (compliance member) is preferably about 20 mm to 40 mm, but is not limited to this.

As described above, according to the medical diagnostic apparatus of the present invention, the shock absorbing member (compliance member) is arranged on the bed, thereby enabling reduction of the burden of the examinee such as oppression or collision, and improvement of comfort, even in a case where the housing and the bed move relative to each other. Additionally, a part of the examinee can be prevented from being caught by the moving mechanism unit, and safety of the examinee can be improved.

In the above description, the bed and the shock absorbing member (compliance member) are formed of different members. However, the present invention is not limited in that composition. Concretely, an edge of the bed, which defines the opening of the bed (the first opening), can be formed of the shock absorbing member (compliance member). In addition, the shock absorbing member (compliance member) is a member which reduces the load on the examinee. That is, the shock absorbing member can be made of softer member than the bed. When a part of the bed (opening edge) is formed of the shock absorbing member (compliance member) as described above, the part of the bed (opening edge) is made of softer member than the other part of the bed (main part which supports the examinee).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-157697, filed on Jul. 13, 2012, which is hereby incorporated by reference herein in its entirety.

The following is a complete listing of the claims in this application, reflects all changes currently being made to the claims, and replaces all earlier versions and all earlier listings of the claims:

1. A medical diagnostic apparatus comprising:
a bed configured to support an examinee in a prone position, and having a first opening in which a part of the examinee is inserted as an object being examined;
a measurement unit having a second opening, which is smaller than said first opening and in which the object is inserted through said first opening;
a moving mechanism configured to move said bed and said measurement unit relative to each other; and
a shock absorbing member disposed between said bed and the examinee,
wherein said shock absorbing member has a portion covering a part of said first opening at least in a direction along which said bed and said measurement unit move relative to each other, and is arranged such that said covering portion does not cover said second opening.

2. The medical diagnostic apparatus according to claim 1, wherein said measurement unit has a chest wall supporting member configured to support a chest wall of the examinee, and a holding unit configured to hold the object inserted through said second opening.

3. The medical diagnostic apparatus according to claim 1, wherein said covering portion of said shock absorbing member extends into the first opening at least 10 mm.

4. The medical diagnostic apparatus according to claim 1, wherein said shock absorbing member is arranged on all of said bed.

5. The medical diagnostic apparatus according to claim 1, wherein said shock absorbing member is 20 mm to 40 mm in thickness.
